(19) United States
(12) Patent Application Publication
(10) Pub. No.: US 2021/0106417 A1
(43) Pub. Date: Apr. 15, 2021

(54) Ligament Fixator in Knee Surgery

(71) Applicant: Ming Ni, Beijing (CN)

(72) Inventors: Ming Ni, Beijing (CN); Jiying Chen, Beijing (CN)

(21) Appl. No.: 16/607,985

(22) PCT Filed: Dec. 12, 2018
(86) PCT No.: PCT/CN2018/120514

§ 371 Date: Oct. 24, 2019

(30) Foreign Application Priority Data
Feb. 13, 2018 (CN) 201820255993.0
Feb. 13, 2018 (CN) 201820255994.5

(51) Int. Cl. A61F 2/08; A61B 17/064; A61B 17/80; A61B 17/84; A61B 17/86

(52) U.S. Cl. CPC A61F 2/0811; A61B 17/0642; A61B 17/8061; A61B 17/809; A61F 2002/0858; A61B 17/86; A61F 2002/0817; A61F 2220/0016; A61F 2002/0888; A61B 17/844

(57) ABSTRACT

A ligament fixator which includes a fixing plate and a plurality of fastening nails, wherein, the fastening nails are respectively located on the edge of the same side face of the fixing plate; when the fixator is used for fixing the collateral ligaments of the knee, the fastening nails are knocked in and fixed in the femoral condyle so that the collateral ligaments are tightly pressed between the fixing plate and the femoral condyle, and the fixing plate completely touches the attachment points of the collateral ligament to form large compaction area, uniform compaction force and excellent compaction effect, thereby guaranteeing operative effect and recovery effect of a patient; when being used for preventing the injury of the patellar tendon at the tibial tubercle, the fastening nails are knocked in and fixed in the tibia so that the fixing plate is located outside the patellar tendon.

### TECHNICAL FIELD

This application pertains to the technical field of medical surgical instruments, and particularly relates to a ligament fixator in a knee surgery.

### BACKGROUND

In artificial total knee replacement, the femoral condylar attachment points of the femoral condylar of the collateral ligament need to be upwardly moved (slip osteotomy) if there are situations that the anterolateral rotation of the knee is unstable or the chronic knee lateral ligament is relaxed. In addition, for osteoporotic patients, the avulsion of the attachment points of the collateral ligament may occur during the surgical exposure. Under the above situations, the attachment points of the collateral ligament all need to be fixed in the shallow bone groove excavated at the planned position to reconstruct the anatomy and function of the collateral collateral ligaments. However, because screws are only used for positioning, the attachment points of the collateral ligaments are compacted only through the head of the screw so that the compaction area is small, and the attachment points of the collateral ligament can not be uniformly stressed, thereby resulting in unsatisfactory fixation effect to affect surgical effect and recovery of patients.

Many knee surgeries are all performed in an area near the tibial tubercle. In the surgical procedure, surgical instruments easily touch the tibial tubercle which are covered with patellar tendons. Because the tibial tubercle is the starting point of the patellar tendon, if the patellar tendon at the tibial tubercle is damaged, it will greatly influence the function of the patellar tendon. The existing knee surgery does not provide any protection measures for the tibial tubercle so as to result in the risk of injuring the patellar tendon at the tibial tubercle during the surgical procedure.

### SUMMARY

In order to overcome the problems existing in the related technology at least to a certain degree, the objective of this application is to provide a ligament fixator in a knee surgery, which can solve the problems that the attachment points of the collateral ligament are compacted only through the head of the screw so that compaction area is small, the attachment points of the collateral ligament can not be uniformly stressed, and the patellar tendon at the tibial tubercle is easily damaged in the existing knee surgery.

This application provides a ligament fixator in a knee surgery for fixing collateral ligaments of a knee or preventing the injury of the patellar tendon at the tibial tubercle, comprising a fixing plate and a plurality of fastening nails, wherein, the fastening nails are respectively located at the edge of the same side face of the fixing plate; when the fastening nails are knocked in and fixed in the femoral condyle, the fixing plate (1) is abutted against the femoral condyle; when the fastening nails are knocked in and fixed in the tibia, the fixing plate is located outside the patellar tendon.

Preferably, the fixing plate is set as a rectangular platy structure, four fastening nails are set and respectively located at four corners of the platy structure.

Preferably, the middle of the fixing plate is provided with a mounting hole penetrating through two side faces of the fixing plate, and a screw assembly for fixing the femoral condyle is penetrated into the mounting hole.

Preferably, the screw assembly is set as an expansion screw assembly, including an expansion screw having an inner cavity and an inner core embedded in the inner cavity; when the inner core moves from a first position to a second position, the expansion screw is expanded and abutted against the femoral condyle.

Preferably, the inner cavity is divided into a first chamber and a second chamber along the axis direction, the inner core is rotatably provided in the first chamber, the inner diameter of the second chamber is smaller than the outer diameter of the inner core, the side wall of the expansion screw surrounding the second chamber is provided with a gap, and the gap extends along the axis direction of the expansion screw and penetrates through the inside and outside of the side walls; when the inner core rotates along the first direction, the inner core moves toward the second chamber and expands the expansion screw.

Preferably, one end of the inner core close to the second chamber is set as a conical shape, and the other end is provided with a rotary knob.

Preferably, two groups of mounting holes and screw assemblies are set.

Preferably, the diameter of one end of one side of the mounting hole which is close to fixing plate and is provided with the fastening nail is smaller than that of the other end.

Preferably, the side wall of the mounting hole is a sphere-arc shaped wall.

Preferably, the fastening nail is of a conical shape, the part of the fastening nail close to the fixing plate is provided with a plurality of ring-shaped grooves distributed along the axis direction.

Preferably, the tip of the fastening nail is inclined toward the mounting hole.

Preferably, the fixing plate is set as an inferior arc shape formed by bending the rectangular plate along the length direction, and two groups of fastening nails are set and symmetrically provided at two ends of the inner side face of the fixing plate.

Preferably, the length direction of the fastening nail is parallel to the radial direction of the center of the fixing plate.

Preferably, the edge of the inner side face of the fixing plate is provided with a plurality of compaction bumps, and one end of the compaction bump far away from the fixing plate is set as a sphere-arc shape.

Preferably, the middle of the outer side face of the fixing plate is provided with a mounting plate, the mounting plate is perpendicular to the width direction of the fixing plate, and two sides of the mounting plate are oppositely provided with connection blind holes.

Preferably, the connection blind hole is an oblong hole, and the length direction of the oblong hole is perpendicular to the width direction of the fixing plate.

Preferably, the fastening nail is of a triangular pyramid shape.

Preferably, two long sides of the fixing plate are oppositely provided with grooves for extension and clamping of clamping pliers.

Preferably, the corners of the fixing plate are all rounded.

Preferably, the fixing plate is also provided with a plurality of cylinder holes penetrating through the two side faces of the fixing plate, and the fixing plate and the fastening nail are of an integrated structure.

The technical solution of this application includes the following beneficial effects:

When the ligament fixator in a knee surgery is used for fixing the collateral ligament of the knee, the fixing nail is knocked in and fixed in the femoral condyle so that the fixing plate is abutted against the femoral condyle, and then the collateral ligament is tightly pressed between the fixing plate and the femoral condyle to achieve the fixation of the collateral ligament; specifically, when in use, after the upward movement degree of the ligament is determined, the bone cortex at the upward movement end point is chiseled out and the shallow bone groove is formed. The groove holes corresponding to the positions of the plurality of fastening nails are chiseled out in the shallow bone groove to fix the attachment points of the collateral ligament in the shallow bone groove, and then the fastening nails are respectively aligned with the groove holes and the force is applied downward so that the fixing plate tightly presses the collateral ligament to form the fixation of the attachment points of the collateral ligament. The fixing plate fully touches the attachment points of the collateral ligament so that the compaction area is large, compaction is even, and the compaction effect is excellent, thus guaranteeing the surgical effect and recovery effect of patients.

When the ligament fixator in a knee surgery is used for preventing the injury of the patellar tendon injury at the tibial tubercle, the fastening nail is knocked in and fixed in the tibia so that the fixing plate is located outside the patellar tendon, thus playing a protective role in the patellar tendon; specifically, when in use, the positions of the patellar tendon and the fastening nail are determined so that the fastening nail is located at both sides of the patellar tendon, downwardly pushed and pressed, so that the tip of the fastening nail is knocked into the tibia until the inner side of the fixing plate is abutted against the patellar tendon. The fixing plate shields the patellar tendon at the tibial tubercle, thereby avoiding other surgical instruments to direct touch the patellar tendon at the tibial tubercle during the surgical procedure, and strengthening the fixation of the stop of the patellar tendon to prevent avulsion. Therefore, the patellar tendon at the tibial tubercle can be effectively protected and the damage to the patellar tendon at the tibial tubercle is avoided.

It should be understood that the above general description and detailed description hereinafter are only illustrative and exemplary, and cannot limit this application.

### BRIEF DESCRIPTION OF THE DRAWINGS

Drawings here are incorporated into the specification and constitute one part of the specification, show embodiments meeting this application, and are used for explaining the principle of this application together with the specification.

In order to more clearly illustrate embodiments of the present utility model and the technical solution in the prior art, embodiments or drawings required to be used in the description of the prior art will be simply introduced. Apparently, the drawings in the following description are only some embodiments of the present utility model, and those of ordinary skill in the art can also obtain other drawings according to these drawings without any creative efforts.

FIG. 1 is a front view of the present utility model according to a first exemplary embodiment;

FIG. 2 is a top view of the present utility model according to a first exemplary embodiment;

FIG. 3 is an A-A cross-sectional view in FIG. 2;

FIG. 4 is a front view of the present utility model according to a second exemplary embodiment;

FIG. 5 is a top view of the present utility model according to a second exemplary embodiment;

FIG. 6 is a structure diagram of an expansion screw assembly according to some exemplary embodiments;

FIG. 7 is a structural diagram of an inner core according to some exemplary embodiments;

FIG. 8 is a front view of the present utility model according to a third exemplary embodiment;

FIG. 9 is a B-B cross-sectional view in FIG. 8;

FIG. 10 is a top view of the present utility model according to a third exemplary embodiment;

FIG. 11 is a C-C cross-sectional view in FIG. 10;

FIG. 12 is a side view of the present utility model according to a third exemplary embodiment.

In drawings, 1-fixing plate, 11-mounting hole, 12-cylinder hole, 13-groove, 14-compaction bump, 15-mounting plate, 151-connection bind hole, 2-fastening nail, 21-ring-shaped groove, 31-expansion screw, 32-inner core

### DESCRIPTION OF THE EMBODIMENTS

Here, exemplary embodiments will be described in detail, and their examples are represented in the drawings. When the following description concerns the drawings, unless otherwise indicated, the same numbers in different drawings represent the same or similar elements. The embodiments described in the following exemplary embodiments do not represent all embodiments consistent with this application. On the contrary, they are only examples of devices or methods consistent with some aspects of this application.

The embodiment provides a ligament fixator in a knee surgery. When the ligament fixator is used for fixing the collateral ligament of the knee, the fixing nail is knocked in and fixed in the femoral condyle so that the fixing plate is abutted against the femoral condyle, and then the collateral ligament is pressed tightly between the fixing plate and the femoral condyle to achieve the fixation of the collateral ligament, the fixing plate fully touches the attachment points of the collateral ligament, so that the compaction area is large, compaction is even, and the compaction effect is excellent; when the ligament fixator is used for preventing injury of the patellar tendon injury at the tibial tubercle, the fastening nail is knocked in and fixed in the tibia so that the fixing plate is located outside the patellar tendon, thus playing a protective role in the patellar tendon.

For making the objective, technical solution and advantages of the present utility model more clear, the technical solution of the present utility model will be described in detail below. Obviously, the described embodiments are only a part of embodiments of the present utility model but not all the embodiments. Based on the embodiments of the present utility model, other embodiments made by those of ordinary skill in the art without creative efforts are all included within the protection scope of the present utility model.

Next, embodiments will be explained with reference to drawings. In addition, embodiments mentioned below do not limit the contents of the utility model described by claims. In addition, the entire content of the composition represented in the following embodiments is not limited to those necessary as the solutions of the utility model described in the claims.

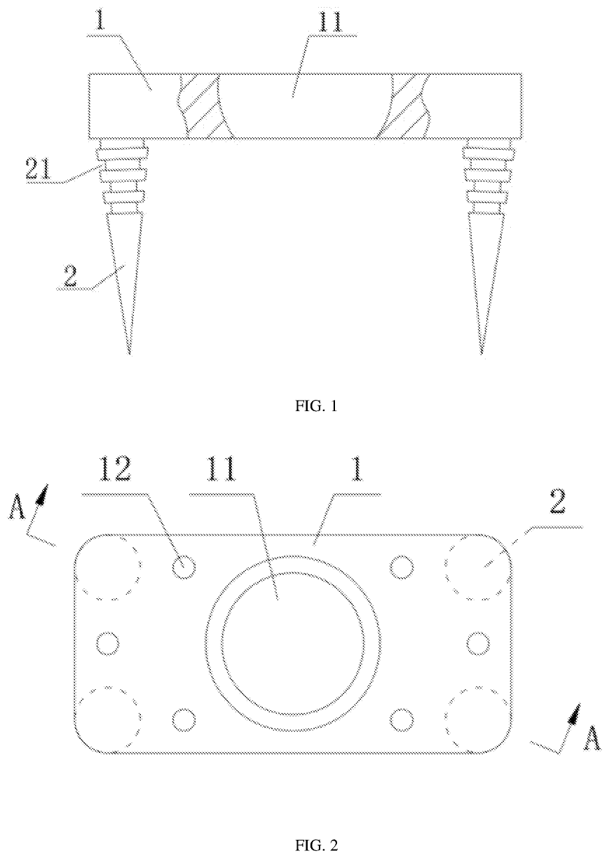

Referring to FIG. 1-FIG. 2, this embodiment provides a ligament fixator in a knee surgery for fixing collateral ligaments of a knee or preventing the injury of the patellar tendon at the tibial tubercle, comprising a fixing plate 1 and a plurality of fastening nails 2, wherein, the plurality of fastening nails 2 are respectively located on the edge of the same side face of the fixing plate 1, in this way, the fixing plate 1 is connected and fixed by the plurality of fastening nails 2, thereby facilitating the promotion of the connection stability and compactibility of the fixing plate 1.

It is understood that the shape of the fixing plate 1 may be square, or round or triangle, and meanwhile the quantity of the fastening nails 2 is determined according to the shape of the fixing plate 1, the quantity of the fastening nails 2 may be 2 or 4, depending on specific circumstances; the fastening nails 2 are located on the edge of the fixing plate 1, which facilitates the promotion of connection balance of the fixing plate 1, or the middle of the fixing plate 1 is empty, which facilitates the better compaction of the collateral ligament or protection of the patellar tendon at the tibial tubercle.

When the ligament fixator in a knee surgery is used for fixing the collateral ligament of the knee, the fixing nail 2 is knocked in and fixed in the femoral condyle so that the fixing plate 1 is abutted against the femoral condyle, and then the collateral ligament is pressed tightly between the fixing plate 1 and the femoral condyle to achieve the fixation of the collateral ligament; specifically, when in use, after the upward movement degree of the ligament is determined, the cortex of the bone at the upward movement end point is chiseled out and the shallow bone groove is formed. The groove holes corresponding to the positions of the plurality of fastening nails 2 are chiseled out in the shallow bone groove to fix the attachment points of the collateral ligament in the shallow bone groove, and then the fastening nails 2 are respectively aligned with the groove holes and the force is applied downward so that the fixing plate 1 tightly presses the collateral ligament to form the fixation of the attachment points of the collateral ligament. The fixing plate 1 fully touches the attachment points of the collateral ligament, so that the compaction area is large, compaction is even, and the compaction effect is excellent, thus guaranteeing the surigical effect and recovery effect of patients.

When the ligament fixator in a knee surgery is used for preventing the injury of the patellar tendon at the tibial tubercle, the fastening nail 2 is knocked in and fixed in the tibia so that the fixing plate 1 is located outside the patellar tendon, thus playing a protective role in the patellar tendon; specifically, when in use, the positions of the patellar tendon and the fastening nail 2 are determined so that the fastening nail 2 is located at both sides of the patellar tendon, downwardly pushed and pressed, so that the tip of the fastening nail 2 is knocked into the tibia until the inner side of the fixing plate 1 is abutted against the patellar tendon. The fixing plate 1 shields the patellar tendon at the tibial tubercle, thereby avoiding other surgical instruments to directly touch the patellar tendon at the tibial tubercle during the surgical procedure, and strengthening the fixation of the stop of the patellar tendon to prevent avulsion. Therefore, the patellar tendon at the tibial tubercle can be effectively protected and the damage to the patellar tendon at the tibial tubercle is avoided.

In some embodiments, the fixing plate 1 is of a rectangular platy structure. As shown in FIG. 2, four fastening nails 2 are set and respectively provided at four corners of the platy structure, in this way, multidirectional fixation can be achieved, promotion of fixation stability and compaction of the fastening nails 2 is facilitated, and the compaction area of the collateral ligament compacted by the fixing plate 1 is facilitated and the compaction force is improved. Where, the four fastening nails 2 are located at the same side face of the fixing plate 1. For ease of description, the two side faces of the fixing plate 1 are respectively defined as an inner side face and an outer side face, and the side face having the fastening nail 2 is the inner side face.

The middle of the fixing plate 1 is provided with a mounting hole 11 penetrating through the inner side face and the outer side face of the fixing plate 1, the mounting hole 11 is used for mounting a screw assembly to achieve the detachable connection of the screw assembly, the screw assembly is used for fixing the femoral condyle so as to fix the fixing plate 1 on the femoral condyle, in this way, under the effect of the screw assembly, the connection compactibility of the fixing plate 1 can be promoted, and the promotion of the compaction force of the fixing plate 1 to the collateral ligament is facilitated.

In some preferred embodiments, the screw assembly is set as an expansion screw assembly. The expansion screw assembly can be expanded after being knocked, and abutted against the femoral condyle so as to further strengthen the fixation of the collateral ligament. As shown in FIG. 6, the expansion screw assembly includes an expansion screw 31 and an inner core 32, wherein, the expansion screw 31 is provided with an inner cavity extending along the axis direction, and the inner core 32 is embedded in this inner cavity; when the inner core 32 moves from the first position to the second position, the expansion screw 31 is expanded so that the outer diameter of the partial outer wall of the expansion screw 31 is enlarged, and then the expansion screw 31 is abutted against the groove holes excavated on the femoral condyle, thereby facilitating the tight fixation of the fixing plate 1 on the femoral condyle. Where, the first position and the second position are both positions where the inner core is located in the inner cavity. In this way, the expansion screw assembly is simple in structure, can prevent the release of the fixing plate 1 and the femoral condyle, and further strengthens the stability of the fixing plate 1.

Where, the inner cavity of the expansion screw 31 is divided into a first chamber and a second chamber along the axis direction, the inner core 32 can rotatably provided in the first chamber, and the inner diameter of the second chamber is smaller than the outer diameter of the inner core 32; the side wall of the expansion screw 31 surrounding the second chamber is provided with a gap, and the gap divides the side wall into a plurality of parts along the axis direction, namely, the gap extends along the axis direction of the expansion screw 31 and penetrates through the inside and outside of the side wall; in this way, when the inner core 32 rotates along the first direction, the inner core 32 moves toward the direction of the second chamber and extends into the second chamber; because the inner diameter of the second chamber is smaller than the outer diameter of the inner core 32, the expansion screw 31 located in the second chamber is expanded and then abutted against the groove hole of the femoral condyle, in this way, rotation moves the inner core 32 and drives the expansion screw 31 to be expanded, and therefore operation is convenient, and installation is easy.

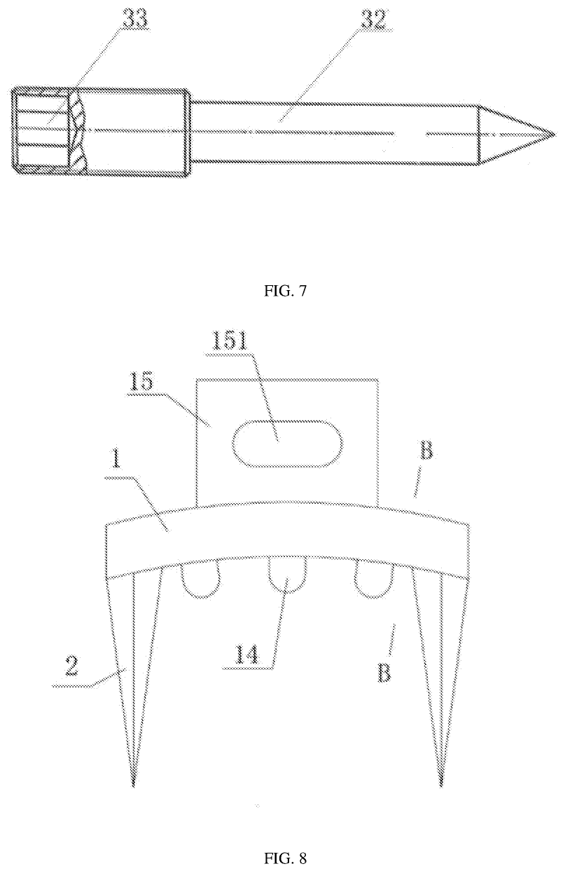

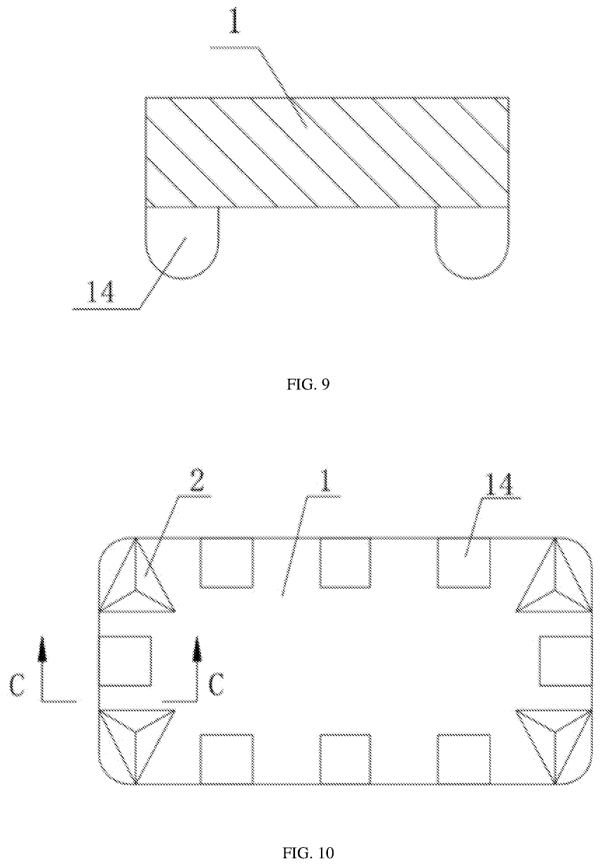

In order to make the inner core 32 easily extend into the second chamber, one end of the inner core 32 close to the second chamber is set as a conical shape, and the other end of the inner core 32 is provided with a rotary knob, as shown in FIG. 7. In this way, installation is time saving and labor saving, and is convenient and fast.

In order to further promote the fixation stability and tightness of the fixing plate 1 and the femoral condyle; two groups of the fixing holes 11 and the screw assembles are arranged, which are symmetrically provided along the center of the fixing plate 1, thereby facilitating maintaining the installation balance.

In some preferred embodiments, the diameter of the mounting hole 11 located at one end of the inner surface of the fixing plate 1 is smaller than that at the other end, so that when the screw assembly passes through the mounting hole 11, the mounting hole 11 can accommodate the head of the screw assembly, so that the head of the screw assembly is sunk into the mounting hole 11, and the overall combination thickness of the head of the screw assembly and the fixing plate 1 is reduced.

Where, the side wall of the mounting hole 11 is set as a sphere-arc shaped wall, and the screw assembly also uses the specifications matching the head with the shape of the side wall of the mounting hole 11. The sphere-arc shaped wall can increase the touch area between the head of the screw and the side wall of the installation hole 11 without increasing the depth of the mounting hole 11, so as to enhance the fixation effect.

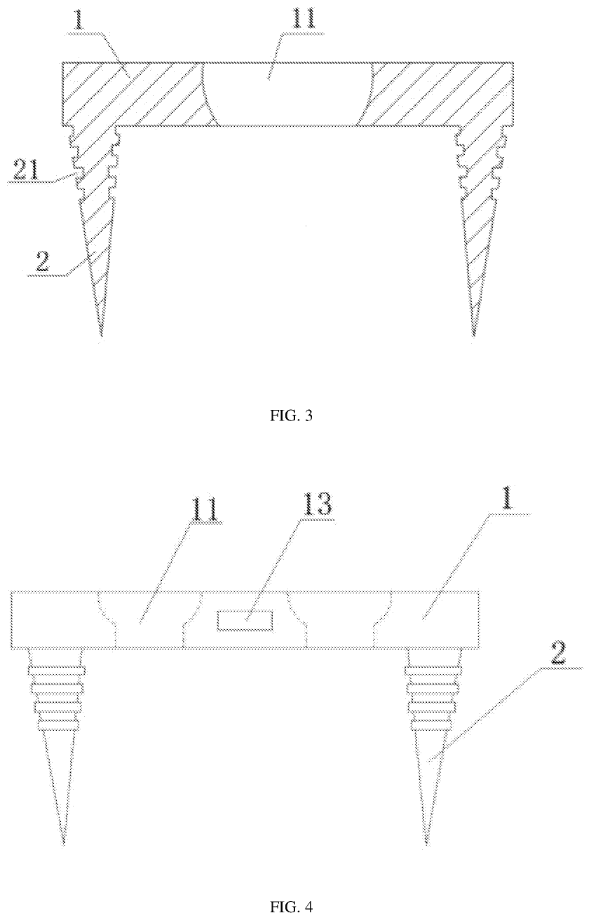

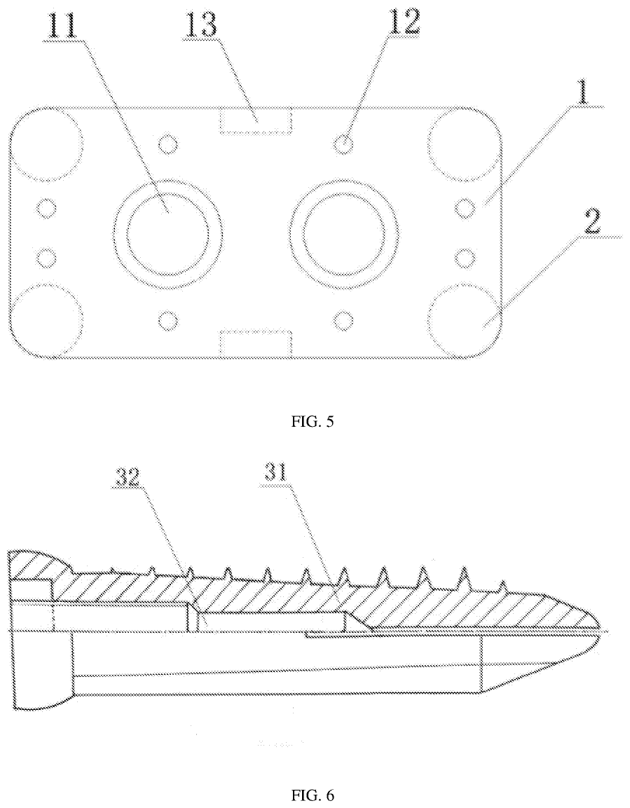

The fastening nail 2 is of a conical shape. As shown in FIG. 3, the part of the fastening nail 2 close to the fixing plate 1 is provided with a plurality of ring-shaped grooves 21 distributed along the axis direction. In this way, during the recovery of patients, the bone grows and healed and forms interlocking with the ring-shaped grooves 21, so as to play a fixation role in the fixing plate 1, and effectively ensure the fixation of the attachment points of the collateral ligament.

The tips of the four fastening nails 2 are inclined toward the mounting hole 11, so that an included angle is formed between the axis of the fastening nail 2 and the axis of the mounting hole 11, and the fixation effect of the collateral ligament of the knee can be better achieved.

Particularly, included angles of 2 degrees are formed between the axis of the four fastening nails 2 and the axis of the mounting hole 11, so that it is ensured that not only the fastening nails 2 can smoothly enter the bone but also better fixation effect can be generated.

In some embodiments, the fixing plate 1 is set as an inferior arc-shaped structure formed by bending the rectangular plate along the length direction, as shown in FIG. 8, where the two groups of fastening nails 2 are set and symmetrically provided at both ends of the inner side face of the fixing plate 1, namely, the two groups of fastening nails 2 are symmetrically provided in the arc-shaped direction of the inner side of the fixing plate 1 (namely, the length direction of the fixing plate 1). The fastening nails 2 are located at both ends of the fixing plate 1, which can take good positioning effect without making the end of the fixing plate 1 beyond the fastening nail 2, thus avoiding the obstruction of the activities of other surgical instruments. Specifically, four fastening nails 2 can be set and respectively positioned at four corners of the fixing plate 1, thereby taking better positioning and protection effect.

It should be noted that the arc radius of the inner side of the fixing plate 1 can be set as 38 mm so as to be adapted to the arc degree of the tibial tubercle to the greatest extent in order to ensure a good protective effect; the distance between the two groups of fastening nails 2 can be set as 17 mm, which is adapted to the width of the patellar tendon at the tibial tubercle. When the patellar tendon is protected, the patellar tendon is accommodated between the two groups of fastening nails 2 to form an effective protection on the patellar tendon; meanwhile, in order to adapt to the shape of the tibia at the tibial tubercle so as to form a fixation between the fastening nail 2 and the tibia, the length of the fastening nail 2 can be set as 10 mm so that the tip of the fastening nail 2 can be knocked into the surface of the tibia to prevent the displacement of the fixing plate 1.

The length directions of the two groups of fastening nails 2 are both parallel to the radial direction of the center of the fixing plate 1, which can ensure the fixation balance of the fixing plate 1, thus effectively playing a protective role.

In some preferred embodiments, in order to avoid that while the fixing plate 1 protects the patellar tendon, the internal side of the fixing plate 1 completely presses the surface of the patellar tendon to result in a fact that the blood flow of the patellar tendon is poor, the edge of the inner side of the fixing plate 1 is also provided with a plurality of compaction bumps 14. The height of the compaction bump 14 is 2 mm. When the patellar tendon is protected by the fixing plate 1, the patellar tendon is compressed locally by one end of the compaction bump far away from the fixing plate 1, so that a space is reserved between the surface of the patellar tendon and the inner side of the fixing plate 1, and the fixing plate is completely compacted on the surface of the patellar tendon. Thus, the smooth blood flow of the patellar tendon can be guaranteed, the normal blood supply of the patellar tendon can be guaranteed, and the function of the patellar tendon is not affected any more.

Where, one end of the compaction bump 14 far away from the fixing plate 1 is set as a sphere-arc shape, which ensures the effective compaction of the compaction bump 14 on the surface of the patellar tendon without damaging the patellar tendon.

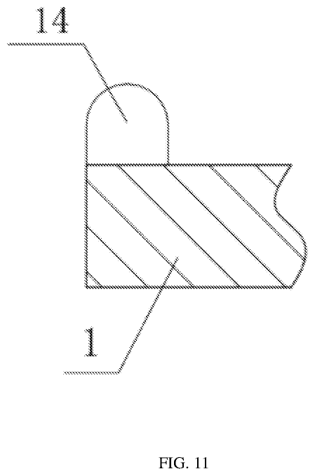

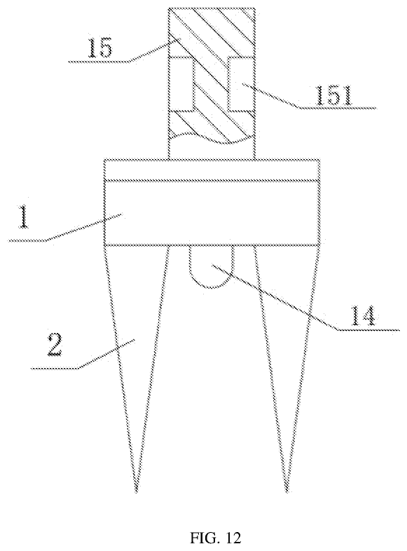

Further, 8 compression bumps 14 are set, as shown in FIG. 10, in which two compaction bumps 14 are symmetrically located in the middle of the two ends of the fixing plate 1 in the arc length direction, and the other six compaction bumps 14 are symmetrically arranged along the width direction of the fixing plate 1 and evenly distributed along the arc length direction of the fixing plate 1. In this way, the patellar tendon can be evenly compacted.

In some preferred embodiments, in order to match with the holding of a holder on the fixing plate 1, the outer side of the fixing plate 1 is provided with a mounting plate 15, and the mounting plate 15 is perpendicular to the width direction of the fixing plate 1, and the two sides of the fixing plate 1 are respectively provided with connection blind holes 151 whose positions correspond. The holder has two holding parts corresponding to the positions of two blind holes 151. When the holder is connected with the mounting plate 15, the two holding parts have the same holding force to hold the mounting plate 15. The two holders are extended into the two blind holes 151 to form the limited position of the mounting plate 15 to avoid slipping.

Further, the connecting blind hole 151 is an oblong hole, and the length direction of the oblong hole is perpendicular to the width direction of the fixing plate 1. The mounting plate 15 is fixed in the middle of the fixing plate 1 so as to form the most stable connection with the holder.

The fastening nail 2 is set in a triangular pyramid shape, so the fastening nail 2 has better structural strength and better positioning effect.

In this embodiment, two long sides of the fixing plate 1 are oppositely provided with two grooves 13. As shown in FIG. 4, the groove 13 is used for extending and holding the holding ends of the holding pliers, thus facilitating the positioning and installation of the fixing plate 1 and improving the promotion of installation efficiency.

The four corners of the fixing plate 1 are all rounded, so as to avoid the damage of prismatic corners on human muscles or soft tissues.

The fixing plate 1 is also provided with a plurality of cylinder holes 12 penetrating through two sides of the fixing plate 1. The cylinder hole 12 can be used for auxiliary positioning in the positioning process of the fixing plate 1, while the cylinder hole 12 can be penetrated into the suture to strengthen or tighten the ligament. The diameter of the cylinder hole 12 is smaller than that of the root of the fixing nail 2 to avoid the influence on the effective compaction area of the fixing plate 1.

The fixing plate 1 and the fastening nail 2 are integrally formed to ensure the overall structural strength. Both the fixing plate 1 and the fastening nail 2 are made of titanium alloy, and their surfaces are sprayed with HA coatings.

It should be noted that, without conflict, the features of the embodiments of the present utility model can be combined with each other. The phrases “first” and “second” herein do not limit the specific sequence, but are only used to distinguish various parts or functions. In the description of the present utility model, it should also be noted that, unless otherwise specified, and defined, the terms “setting”, “installation”, “linking”, “connection” should be understood in a broad sense, such as fixed connection, detachable connection, or integral connection, or mechanical connection, or electrical connection; or direct linking, or linking through an intermediate medium, or communication of insides of the two assemblies. For those of ordinary skill in the art, the specific meaning of the above terms in the present utility model can be understood in specific circumstances.

The above descriptions mentioned are only embodiments of the present utility model, but the protection scope of the present utility model is not limited thereto. Any technician familiar with the technical field can easily conceive changes or substitutions within the scope of the disclosed technology of the present utility model, these changes or substitutions should be covered in the protection scope of the present utility model. Thus, the protection scope of the present utility model should be based on the protection scope of the claims.

It is understood that the same or similar parts of the above-mentioned embodiments can be referred to each other, and contents that are not explained in detail in some embodiments can refer to the same or similar contents in other embodiments.

Although the embodiments of this application have been illustrated and described above, it is understood that the above embodiments are illustrative and cannot be construed as limitations to this application. Persons of ordinary skill in the art may make variations, modifications, replacements and transformation to the above embodiments within the scope of this application.

1. A ligament fixator in a knee surgery for fixing collateral ligaments of a knee or preventing the injury of the patellar tendon at the tibial tubercle, comprising a fixing plate (1) and a plurality of fastening nails (2), wherein, the fastening nails (2) are respectively located at the edge of the same side face of the fixing plate (1); when the fastening nails (2) are knocked in and fixed in the femoral condyle, the fixing plate (1) is abutted against the femoral condyle; when the fastening nails (2) are knocked in and fixed in the tibia, the fixing plate (1) is located outside the patellar tendon.

2. The ligament fixator in a knee surgery according to claim 1, wherein, the fixing plate (1) is set as a rectangular platy structure, four fastening nails (2) are set and respectively located at four corners of the platy structure.

3. The ligament fixator in a knee surgery according to claim 2, wherein, the middle of the fixing plate (1) is provided with a mounting hole (11) penetrating through two side faces of the fixing plate (1), and a screw assembly for fixing the femoral condyle is penetrated into the mounting hole (11).

4. The ligament fixator in a knee surgery according to claim 3, wherein, the screw assembly is set as an expansion screw assembly, including an expansion screw (31) having an inner cavity and an inner core (32) embedded in the inner cavity; when the inner core (32) moves from a first position to a second position, the expansion screw (31) is expanded and abutted against the femoral condyle.

5. The ligament fixator in a knee surgery according to claim 4, wherein, the inner cavity is divided into a first chamber and a second chamber along the axis direction, the inner core (32) is rotatably provided in the first chamber, the inner diameter of the second chamber is smaller than the outer diameter of the inner core (32), the side wall of the expansion screw (31) surrounding the second chamber is provided with a gap, and the gap extends along the axis direction of the expansion screw (31) and penetrates through the inside and outside of the side wall; when the inner core (32) rotates along the first direction, the inner core (32) moves toward the second chamber and expands the expansion screw (31).

6. The ligament fixator in a knee surgery according to claim 5, wherein, one end of the inner core (32) close to the second chamber is set as a tapered shape, and the other end is provided with a rotary knob.

7. The ligament fixator in a knee surgery according to claim 3, wherein, two groups of mounting holes (11) and screw assemblies are set.

8. The ligament fixator in a knee surgery according to claim 3, wherein, the diameter of one end of one side of the mounting hole (11) which is close to the fixing plate (1) and is provided with the fastening nail (2) is smaller than that of the other end.

9. The ligament fixator in a knee surgery according to claim 8, wherein, the side wall of the mounting hole (11) is a sphere-arc shaped wall.

10. The ligament fixator in a knee surgery according to claim 2, wherein, the fastening nail (2) is of a conical shape, the part of the fastening nail (2) close to the fixing plate (1) is provided with a plurality of ring-shaped grooves (21) distributed along the axis direction.

11. The ligament fixator in a knee surgery according to claim 3, wherein, the tip of the fastening nail (2) is inclined toward the mounting hole (11).

12. The ligament fixator in a knee surgery according to claim 1, wherein, the fixing plate (1) is set as an inferior arc shape formed by bending the rectangular plate along the length direction, two groups of fastening nails (2) are set and symmetrically provided at two ends of the inner side face of the fixing plate (1).

13. The ligament fixator in a knee surgery according to claim 12, wherein, the length direction of the fastening nail (2) is parallel to the radial direction of the center of the fixing plate (1).

14. The ligament fixator in a knee surgery according to claim 12, wherein, the edge of the inner side face of the fixing plate (1) is provided with a plurality of compacted bumps (14), and one end of the compacted bump (14) far away from the fixing plate (1) is set as a sphere-arc shape.

15. The ligament fixator in a knee surgery according to claim 12, wherein, the middle of the outer side face of the fixing plate (1) is provided with a mounting plate (15), the mounting plate (15) is perpendicular to the width direction of the fixing plate (1), and two sides of the mounting plate (15) are oppositely provided with connection blind holes (151).

16. The ligament fixator in a knee surgery according to claim 12, wherein, the connection blind hole (151) is an oblong hole, and the length direction of the oblong hole is perpendicular to the width direction of the fixing plate (1).

17. The ligament fixator in a knee surgery according to claim 12, wherein, the fastening nail (2) is of a triangular pyramid shape.

18. The ligament fixator in a knee surgery according to claim 2, wherein, two long sides of the fixing plate (1) are oppositely provided with grooves (13) for extension and clamping of clamping pliers.

19. The ligament fixator in a knee surgery according to claim 2, wherein, the corners of the fixing plate (1) are all rounded.

20. The ligament fixator in a knee surgery according to claim 1, wherein, the fixing plate (1) is also provided with a plurality of cylinder holes (12) penetrating through the two side faces of the fixing plate (1), and the fixing plate (1) and the fastening nail (2) are of an integrated structure.
